(19) United States
(12) Patent Application Publication
(10) Pub. No.: US 2026/0346053 A1
(43) Pub. Date: Nov. 5, 2026

(54) SKIN CARE DEVICE

(71) Applicant: Jeisys Medical Inc., Seoul (KR)

(72) Inventors: Hyoung Moon Kim, Seoul (KR); Dong Hwan Kang, Seoul (KR)

(73) Assignee: Jeisys Medical Inc., Seoul (KR)

(21) Appl. No.: 19/638,852

(22) Filed: Apr. 3, 2026

Related U.S. Application Data
(63) Continuation of application No. PCT/KR2024/015136, filed on Oct. 4, 2024.

(30) Foreign Application Priority Data
Oct. 5, 2023 (KR) 10-2023-0132556
Jan. 18, 2024 (KR) 10-2024-0007845
Sep. 5, 2024 (KR) 10-2024-0120985

(51) Int. Cl. A61N 7/00

(52) U.S. Cl. CPC A61N 7/00; A61N 2007/0034

(57) ABSTRACT

An ultrasound irradiation module configured to irradiate ultrasound energy to a human body. Embodiments include a processor to control an operation of the ultrasound irradiation module. The ultrasound irradiation module can irradiate ultrasound energy for fat proliferation to an irradiation position of the human body.

### CROSS-REFERENCE TO RELATED APPLICATIONS

The present application is a continuation of International Patent Application No. PCT/KR2024/015136, filed on Oct. 4, 2024, which is based upon and claims the benefit of priority to Korean Patent Application Nos. 10-2023-0132556 filed on Oct. 5, 2023, 10-2024-0007845 filed on Jan. 8, 2024, and 10-2024-0120985 filed on Sep. 5, 2024. The disclosures of the above-listed applications are hereby incorporated by reference herein in their entirety and for all purposes.

### BACKGROUND

### 1. Technical Field

The present disclosure relates to a skin care device. More specifically, the present disclosure relates to a skin care device capable of enhancing fat proliferation of a human body by irradiating ultrasound energy for fat proliferation.

### 2. Description of Related Art

Ultrasound refers to a wave having a frequency of 20 KHz or more, a property of penetrating water.

The use of ultrasound in the medical field is most typically in an ultrasound imaging device using transmission and reflection properties of ultrasound. For example, there is a device that obtains a cross-sectional image inside a human body by visualizing time and intensity reflected while ultrasound penetrates inside the human body and passes through respective organs.

In addition, there is a device that burns and removes specific subcutaneous tissues such as tumors in skin by using heat generated by high intensity focused ultrasound (HIFU, High Intensity Focused Ultrasound), or induces degeneration and regeneration of skin tissue to generate skin cosmetic or skin plastic effects such as wrinkle improvement.

Such ultrasound generating devices are used to focus ultrasound at a desired depth, adjust the desired depth, and deliver effective energy to the desired depth without epidermal damage.

However, there is a continuing need for improved ultrasound generating devices for use in connection with a human body.

### SUMMARY

An object of embodiments disclosed in the present disclosure is to provide enhanced fat proliferation of a human body by irradiating ultrasound energy to the human body for fat proliferation.

Problems to be solved by the present disclosure are not limited to the problems mentioned above, and other problems not mentioned will be clearly understood by those skilled in the art from the following description.

A skin care device may include an ultrasound irradiation module configured to irradiate energy; and a controller configured to control an operation of the ultrasound irradiation module, wherein the ultrasound irradiation module may be configured to irradiate ultrasound energy to a human body to enhance fat proliferation at an irradiation position. The irradiation position may be a position where fat is required.

The ultrasound irradiation module may be configured to pre-irradiate ultrasound energy for activation of fat proliferation to a fat pad provided at the irradiation position, and to irradiate ultrasound energy to an individual region, for example a region requiring, desiring or possibly benefiting from fat proliferation (e.g., where fat proliferation may be desired or desirable) among positions of the human body.

The ultrasound energy for fat proliferation may be 0.01 to 0.2 J.

In embodiments, the controller may be configured to further perform wired communication or wireless communication with an acquisition device. For example, the acquisition device may be configured to acquire a preset target depth associated with thickness information for each position of the human body based on preset thickness information for each position of the human body, and the ultrasound irradiation module may be configured to irradiate ultrasound energy to a target depth associated with the thickness information based on thickness information for a position to which ultrasound is to be irradiated, for example from thickness information for each position of the human body acquired from the acquisition device.

In embodiments, the controller may be configured to further perform wired communication or wireless communication with an acquisition device, and when external shape information of the human body acquired through the acquisition device is received, determine a position having fat depression within an individual region requiring or desiring fat proliferation based on the external shape information of the human body. The ultrasound irradiation module may be configured to further irradiate ultrasound energy for fat proliferation to the position having fat depression.

In embodiments, the skin care device may further include a radio-frequency irradiation module configured to irradiate radio-frequency based on a dielectric electrode to the human body; and the ultrasound irradiation module may be configured to irradiate ultrasound energy after the radio-frequency irradiation module irradiates radio-frequency energy to the human body, and further irradiate ultrasound energy for fat proliferation to a position having fat depression within an individual region requiring or desiring fat proliferation.

In embodiments, the ultrasound irradiation module may be configured to further irradiate ultrasound energy for fat proliferation at a perpendicular or inclined angle with respect to a surface of the human body.

In embodiments, the processor may be configured to be connected to an ultrasound imaging device through a wired connection or a wireless connection; and when irradiating ultrasound energy for fat proliferation, control the ultrasound imaging device to capture and display at least one of a focal position and a target depth of the human body.

In embodiments, the processor may be configured to be connected to an ultrasound imaging device through a wired connection or a wireless connection.

In embodiments, the ultrasound irradiation module may be configured to further irradiate ultrasound energy for fat proliferation to the human body based on an image of a Polymer Particle injected into the human body, acquired through the ultrasound imaging device.

In embodiments, the human body may be at least one of a face, a buttock, a breast, and a depressed region.

In embodiments, the irradiated portion of the human body may be at least one of a face, a buttock, a breast, and/or a depressed region. The irradiation module may be configured for irradiation of one or more of these portions of the human body.

The ultrasound irradiation module may be configured to further irradiate ultrasound energy for fat proliferation to at least one of a deep fat layer of the face, a lower region of the buttock, a lower region of the breast, and a lower region of the depressed region.

In embodiments, the ultrasound irradiation module may be configured to, when the human body is a face, irradiate ultrasound energy for fat proliferation to a fat pad provided at an irradiation position of a deep fat layer of the face, and irradiate ultrasound energy for fat proliferation to an individual region requiring or desiring fat proliferation among irradiation positions of the deep fat layer.

In embodiments, the processor may be configured to be connected to an acquisition device through a wired connection or a wireless connection, and when skin skeletal information acquired through the acquisition device is received, determine a position having fat depression within an individual region requiring or desiring fat proliferation based on the skin skeletal information;

The ultrasound irradiation module may be configured to further irradiate ultrasound energy for fat proliferation to the position having fat depression.

In embodiments, when the human body is a face, the skin care device may further include a radio-frequency irradiation module configured to irradiate radio-frequency based on a dielectric electrode to a deep fat layer of the face; and the ultrasound irradiation module may be configured to further irradiate ultrasound energy for fat proliferation to a position having fat depression within an individual region requiring or desiring fat proliferation after the radio-frequency irradiation module irradiates radio-frequency energy to a retaining ligament.

In embodiments, the ultrasound irradiation module may be configured to, when the human body is a face, further irradiate ultrasound energy for fat proliferation at a perpendicular or oblique angle with respect to a surface of a zygomatic arch of the face.

### BRIEF DESCRIPTION OF THE FIGURES

FIG. 1 is a diagram illustrating a configuration of a skin care system according to embodiments of the present disclosure.

FIG. 2 is a diagram illustrating a configuration of the skin care device of FIG. 1.

FIG. 3 is a diagram illustrating, as an example, a process in which the ultrasound irradiation module of FIG. 2 irradiates ultrasound.

FIG. 4 is a diagram illustrating an example of a fat proliferation process of a buttock using the skin care device of FIG. 1.

FIG. 5 is a diagram illustrating an example of a fat proliferation process of a breast using the skin care device of FIG. 1.

FIG. 6 is a diagram illustrating, as another example, a process in which the ultrasound irradiation module of FIG. 2 irradiates ultrasound.

FIG. 7 is a diagram illustrating a fat pad and an individual region requiring or desiring fat proliferation, in accordance with embodiments.

FIG. 8 is a diagram illustrating an example of an occurrence sequence of fat depression during a skin aging process of a human.

FIGS. 9 and 10 are diagrams illustrating examples of superficial facial fat pads and deep facial fat pads.

FIGS. 11 and 12 are diagrams for showing examples of a change in HSP70 expression through cilia regulation of adipose-derived stem cells after ultrasound irradiation of the ultrasound irradiation module of FIG. 2.

FIG. 13 is a diagram for showing examples of a change in cilia length of adipose-derived stem cells after ultrasound irradiation of the ultrasound irradiation module of FIG. 2.

FIGS. 14 and 15 are diagrams for showing examples of a change in adipogenesis through cilia length regulation of adipose-derived stem cells after ultrasound irradiation of the ultrasound irradiation module of FIG. 2, in accordance with embodiments.

FIGS. 16 to 18 are diagrams for showing examples of a change in the number of adipocytes and a thickness of a fat layer through cilia length regulation of adipose-derived stem cells after ultrasound irradiation of the ultrasound irradiation module of FIG. 2.

FIG. 19 is a diagram for showing examples of a change in HSP70 before and after ultrasound irradiation of the ultrasound irradiation module of FIG. 2.

FIG. 20 is a diagram for showing examples of a change in CD166 before and after ultrasound irradiation of the ultrasound irradiation module of FIG. 2.

FIG. 21 is a diagram for showing examples of a change in ARL13B before and after ultrasound irradiation of the ultrasound irradiation module of FIG. 2.

FIG. 22 is a diagram for showing examples of a change in AURKA before and after ultrasound irradiation of the ultrasound irradiation module of FIG. 2.

FIG. 23 is a diagram for showing examples of a change in CEBPα before and after ultrasound irradiation of the ultrasound irradiation module of FIG. 2.

FIG. 24 is a diagram for showing examples of a change in PPARγ before and after ultrasound irradiation of the ultrasound irradiation module of FIG. 2.

### DETAILED DESCRIPTION

Throughout the present disclosure, the same reference numerals denote the same components. The present disclosure does not necessarily describe all elements of all embodiments, and certain general contents in the technical field to which the present disclosure belongs or overlapping contents between the embodiments may be omitted. Terms such as “unit, module, member, and block” used in the specification may be implemented as software or hardware, and according to embodiments, a plurality of “units, modules, members, and blocks” may be implemented as a single component, or a single “unit, module, member, and block” may include a plurality of components.

Throughout the specification, when a part is “connected” to another part, it includes not only a case where the part is directly connected but also a case where the part is indirectly connected, and an indirect connection may include a connection through a wireless communication network.

In addition, when a part “includes” a certain component, it means that other components may be further included rather than excluding other components unless specifically stated otherwise.

Throughout the specification, when a member is positioned “on” another member, it includes not only a case where the member is in contact with the other member but also a case where another member exists between the two members.

Terms such as first and second are used to distinguish one component from another component, and the components are not limited by the above terms.

A singular expression includes a plural expression unless the context clearly indicates otherwise.

In each step, identification symbols may be used for convenience of description and do not necessarily describe the order of the steps, and the steps may be performed in a different order from the stated order unless a specific order is clearly described in context.

Hereinafter, an exemplary principle of operation and exemplary embodiments of the present disclosure will be described with reference to the accompanying drawings.

In the present specification, the controller of the skin care device according to the present disclosure includes all various devices capable of performing arithmetic processing and providing results to a user. For example, the controller of the skin care device according to the present disclosure may include all of a computer, a server device, and a portable terminal, or may be in any one form.

The computer may include at least one of, for example, a notebook, a desktop, a laptop, a tablet PC, and a slate PC equipped with a web browser (WEB Browser).

The server device is a server that processes information by performing communication with an external device, and may include at least one of an application server, a computing server, a database server, a file server, a mail server, a proxy server, and a web server.

The portable terminal is, for example, a wireless communication device ensuring portability and mobility, and may include at least one handheld-based wireless communication device among PCS (Personal Communication System), GSM (Global System for Mobile communications), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access), WiBro (Wireless Broadband Internet) terminal, and a smart phone (Smart Phone), and at least one wearable device among a watch, a ring, a bracelet, an anklet, a necklace, glasses, contact lenses, and a head-mounted-device (HMD).

Embodiments of the skin care device according to the present disclosure include an ultrasound irradiation module configured to irradiate ultrasound energy to a human body; and a processor configured to control an operation of the ultrasound irradiation module. The ultrasound irradiation module may be configured to irradiate ultrasound energy corresponding to fat proliferation to an irradiation position of the human body.

The skin care device can enhance fat proliferation of a human body (e.g., at least portions of a human body) by irradiating ultrasound energy corresponding to fat proliferation to the human body.

Hereinafter, embodiments of the skin care device will be described in detail.

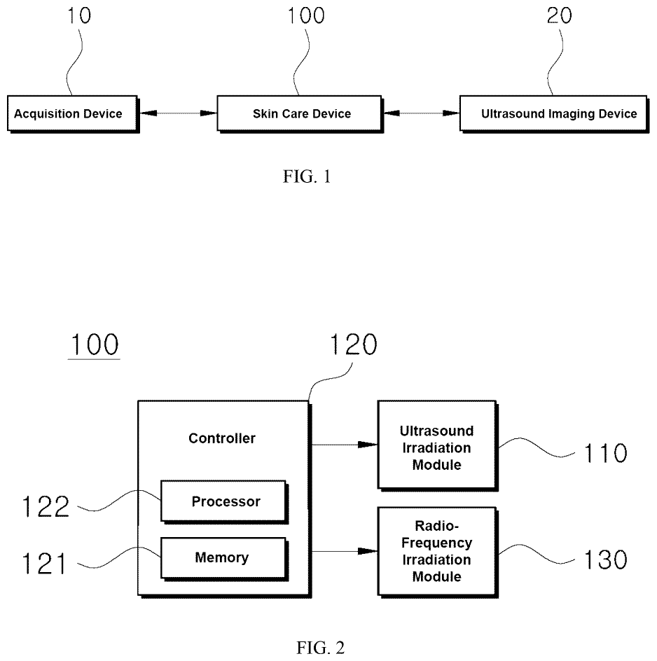

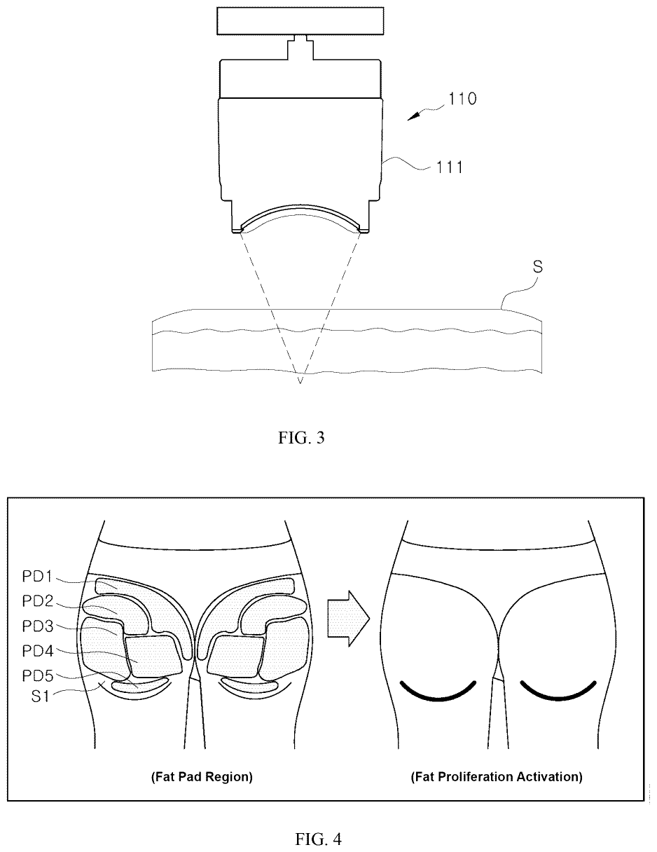

FIG. 1 is a diagram illustrating a configuration of a skin care system according to the present disclosure. FIG. 2 is a diagram illustrating a configuration of the skin care device of FIG. 1. FIG. 3 is a diagram illustrating, as an example, a process in which the ultrasound irradiation module of FIG. 2 irradiates ultrasound.

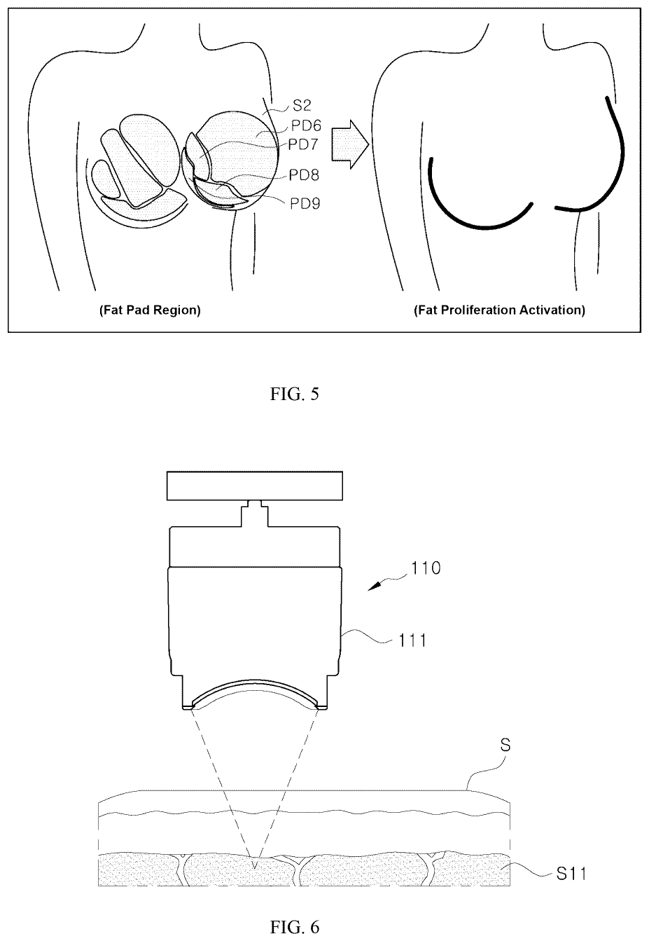

FIG. 4 is a diagram illustrating a fat proliferation process of a buttock using the skin care device of FIG. 1. FIG. 5 is a diagram illustrating a fat proliferation process of a breast using the skin care device of FIG. 1.

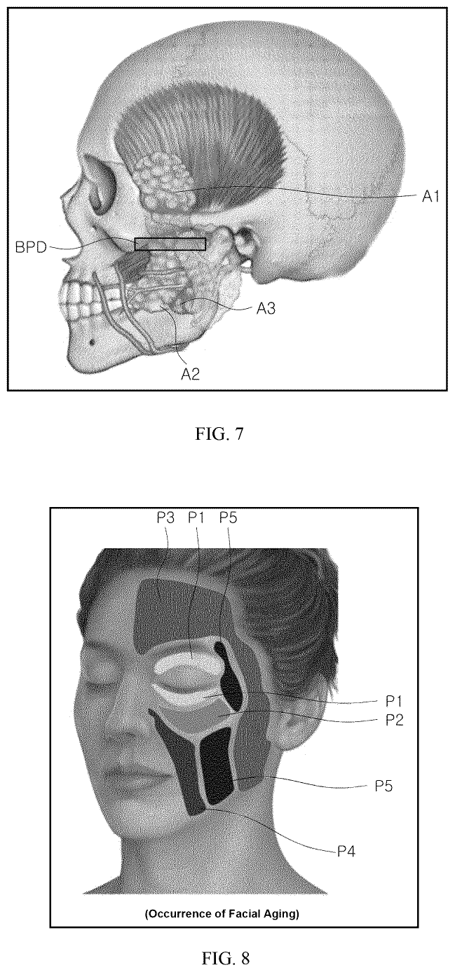

FIG. 6 is a diagram illustrating, as another example, a process in which the ultrasound irradiation module of FIG. 2 irradiates ultrasound. FIG. 7 is a diagram illustrating a fat pad and an individual region requiring, desiring or otherwise benefiting from fat proliferation (e.g., where fat proliferation is desired or may be beneficial).

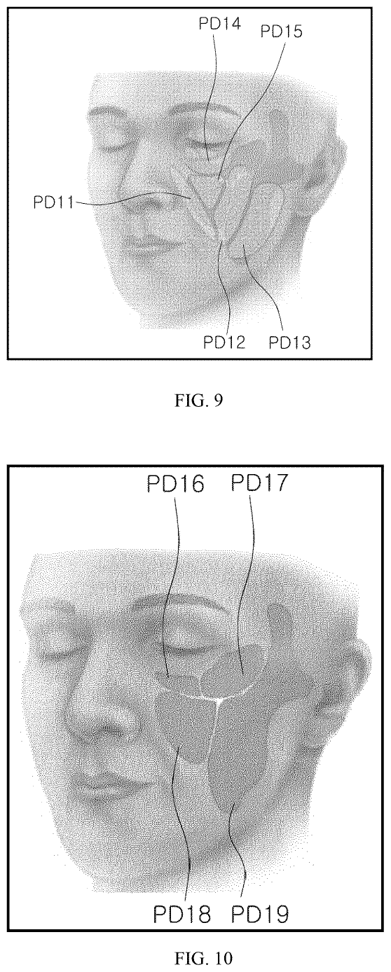

FIG. 8 is a diagram illustrating an occurrence sequence of fat depression during a skin aging process of a human. FIGS. 9 and 10 are diagrams illustrating superficial facial fat pads and deep facial fat pads.

Referring to FIGS. 1 to 10, the skin care device 100 may include an ultrasound irradiation module 110 and a controller 120.

The ultrasound irradiation module 110 includes a transducer 111 may irradiate ultrasound to a human body S while maintaining an ultrasound focal depth. The ultrasound focal depth may be changed according to a user. The ultrasound irradiation module 110 may irradiate ultrasound to the human body S at a changed ultrasound focal depth corresponding to a measured depth.

The ultrasound irradiation module 110 may irradiate ultrasound to at least one of a buttock S1, a breast S2, a face, and a depressed region. An irradiation region of the buttock S1 may, for example, be 20 mm to 10 cm.

In embodiments, the ultrasound irradiation module 110 may irradiate ultrasound to a deep fat layer S11 of a facial region, and the facial region may be under an eye or under a zygomatic arch. The deep fat layer S11 may be a fat layer under an SMAS layer. In embodiments, the depressed region may be another region of the human body where fat proliferation is directly required or desired or beneficial.

The controller 120 may be implemented as a memory 121 configured to store data for an algorithm for controlling operations of components in the device or a program reproducing the algorithm, and at least one processor 122 configured to perform the above-described operations using the data stored in the memory 121. The memory 121 and the processor 122 may be implemented as separate chips, respectively. In embodiments, the memory 121 and the processor 122 may be implemented as a single chip.

The memory 121 may store data supporting various functions of the device, and programs for operations of the controller, may store input/output data, and may store a plurality of application programs (application program or application) driven in the device, data for operations of the device, and instructions. At least some of these application programs may be downloaded from an external server through wireless communication.

The memory 121 may include at least one type of storage medium such as a flash memory type, a hard disk type, an SSD type (Solid State Disk type), an SDD type (Silicon Disk Drive type), a multimedia card micro type, a card type memory (for example, SD or XD memory), RAM (random access memory), SRAM (static random access memory), ROM (read-only memory), EEPROM (electrically erasable programmable read-only memory), PROM (programmable read-only memory), a magnetic memory, a magnetic disk, and an optical disk. The memory 121 may include a database that is separated from the device but connected thereto by wire or wirelessly.

The processor 122 may control an operation of the ultrasound irradiation module 110.

The ultrasound irradiation module 110 may irradiate ultrasound energy corresponding to fat proliferation to an irradiation position of the human body S. For example, the ultrasound irradiation module 110 may irradiate ultrasound energy corresponding to fat proliferation to at least one of the buttock S1, the breast S2, the deep fat layer S11, and the depressed region.

For example, the ultrasound energy may be 0.01 to 0.2 J, preferably 0.1 J to 0.2 J, and most preferably 0.1 J. When the ultrasound energy is less than 0.01 J, an effect of fat proliferation may be insignificant, and when the ultrasound energy exceeds 0.2 J, an effect of fat reduction may be exhibited.

The human body S may be or include a depressed region, and may be, for example, at least one of the buttock S1, the breast S2, and the face, and is not limited thereto, and the human body S may be another region requiring or desiring or possibly benefiting from fat proliferation.

As an example, the ultrasound irradiation module 110 may irradiate ultrasound energy corresponding to fat proliferation to fat pads PD1 to PD5 provided at an irradiation position of the buttock S1 for activation of fat proliferation, and may irradiate ultrasound energy corresponding to fat proliferation to an individual region directly requiring or desiring or benefiting from fat proliferation among irradiation positions of the buttock S1. The fat pads PD1 to PD5 may have the same size or different sizes. For example, the individual region requiring fat proliferation may be a region of the fat pads PD1 to PD5 or a region excluding the fat pads PD1 to PD5. The individual region may be a depressed region of the buttock S1 or may be a lower region of the buttock S1.

In embodiments, the ultrasound irradiation module 110 may irradiate ultrasound energy corresponding to fat proliferation to fat pads PD6 to PD9 provided at an irradiation position of the breast S2 for activation of fat proliferation, and may irradiate ultrasound energy corresponding to fat proliferation to an individual region directly requiring or desiring or benefiting from fat proliferation among irradiation positions of the breast S2. The fat pads PD6 to PD9 may have the same size or different sizes. The individual region requiring fat proliferation may be a region of the fat pads PD6 to PD9 or a region excluding the fat pads PD6 to PD9. The individual region may be a depressed region of the breast S2 or may be a lower region of the breast S2.

In embodiments, the ultrasound irradiation module 110 may irradiate ultrasound energy corresponding to fat proliferation to a fat pad provided at an irradiation position of the deep fat layer S11 for activation of fat proliferation, and may irradiate ultrasound energy corresponding to fat proliferation to an individual region directly requiring or desiring or benefiting from fat proliferation among irradiation positions of the deep fat layer S11. For example, as shown in FIG. 7, the fat pad BPD may refer to fat tissue in a specific part of a body. For example, the fat pad BPD may be a Buccal fat pad, and the individual region requiring or desiring or benefiting from fat proliferation may be at least one of a Temporal extension area A1, a Buccal extension area A2, and a pterygoid extension area A3.

As another example, the processor 122 may be connected to an acquisition device 10 through a wired connection or a wireless connection. The ultrasound irradiation module 110 may irradiate ultrasound energy corresponding to fat proliferation to a target depth of the buttock S1 associated with thickness information for each irradiation position, based on thickness information for each irradiation position of the buttock S1 acquired through the acquisition device 10 before or after irradiation of ultrasound energy.

In embodiments, the processor 122 may be connected to an acquisition device 10 through a wired connection or a wireless connection. The ultrasound irradiation module 110 may irradiate ultrasound energy corresponding to fat proliferation to a target depth of the breast S2 associated with thickness information for each irradiation position, based on thickness information for each irradiation position of the breast S2 acquired through the acquisition device 10 before or after irradiation of ultrasound energy.

In embodiments, the processor 121 may be connected to an acquisition device 10 through a wired connection or a wireless connection. The ultrasound irradiation module 110 may irradiate ultrasound energy corresponding to fat proliferation to a target depth of the deep fat layer S11 associated with thickness information for each irradiation position, based on thickness information for each irradiation position of the deep fat layer S11 acquired through the acquisition device 10 before or after irradiation of ultrasound energy.

Accordingly, since there may be a difference in depth for at least one of the buttock S1, the breast S2, and the deep fat layer S11 for each person, embodiments may irradiate ultrasound energy corresponding to fat proliferation to fat pads PD1 to PD5, PD6 to PD9, and BPD provided at irradiation positions of at least one of the buttock S1, the breast S2, and the deep fat layer S11 for each person, for activation of fat proliferation, based on thickness information for each irradiation position acquired for each person, and may irradiate ultrasound energy corresponding to fat proliferation to an individual region directly requiring or desiring or benefiting from fat proliferation among irradiation positions of at least one of the buttock S1, the breast S2, and the deep fat layer S11 for each person.

As another example, the processor 122 may be connected to an ultrasound imaging device 20 through a wired connection or a wireless connection. When irradiating ultrasound energy corresponding to fat proliferation to at least one of the buttock S1, the breast S2, and the deep fat layer S11, the ultrasound irradiation module 110 may further control the ultrasound imaging device 20 such that at least one of a focal position and a target depth of at least one of the buttock S1, the breast S2, and the deep fat layer S11 is captured and displayed.

Accordingly, the present disclosure may capture and display a surrounding position of at least one of a buttock, a breast, and a zygomatic arch, and since there may be a difference in at least one of a focal position and a target depth of the buttock S1, the breast S2, and the deep fat layer S11 for each person, may accurately capture and display at least one of a focal position and a target depth of the buttock S1, the breast S2, and the deep fat layer S11 for each person. In embodiments, a user may check at least one of a focal position and a target depth of the buttock S1, the breast S2, and the deep fat layer S11 for each person.

As another example, the processor 122 may be connected to an acquisition device 10 through a wired connection or a wireless connection, and when external shape information of the buttock S1 or the breast S2 acquired through the acquisition device 10 is received, may further determine a position having fat depression within an individual region based on the external shape information of the buttock S1 or the breast S2. The ultrasound irradiation module 110 may further irradiate ultrasound energy corresponding to fat proliferation to the position having fat depression.

As another example, the processor 121 may be connected to an acquisition device 10 through a wired connection or a wireless connection, and when skin skeletal information acquired through the acquisition device 10 is received, may further determine a position having fat depression within an individual region based on the skin skeletal information. The ultrasound irradiation module 110 may further irradiate ultrasound energy corresponding to fat proliferation to the position having fat depression.

As shown in FIG. 8, an occurrence sequence of fat depression during a skin aging process of a human may proceed in an order of P1, P2, P3, P4, and P5.

Accordingly, the ultrasound irradiation module 110 may irradiate ultrasound energy corresponding to fat proliferation to the fat pad BPD for activation of fat proliferation, and as shown in FIG. 9, may irradiate ultrasound energy corresponding to fat proliferation to at least one fat pad directly requiring or desiring or benefiting from fat proliferation among superficial facial fat pads PD11 to PD15 and deep facial fat pads PD16 to PD19 corresponding to at least one of P1, P2, P3, P4, and P5 having fat depression within an individual region.

In order to perform volume-up lifting that does not sag due to gravity, radio-frequency irradiation based on a dielectric electrode may be required or provide benefits. The radio-frequency irradiation based on a dielectric electrode can perform volume-up lifting for a face. Such volume-up lifting for a face can contract and strengthen a retaining ligament that maintains a structure and a form of a skin layer.

That is, the skin care device 100 according to the present disclosure may further include a radio-frequency irradiation module 130. Here, the radio-frequency irradiation module 130 may irradiate radio-frequency based on a dielectric electrode to at least one of the buttock S1, the breast S2, and the deep fat layer S11. For example, the dielectric electrode may be a monopolar type electrode or a bipolar type electrode.

The ultrasound irradiation module 110 may further irradiate ultrasound energy corresponding to fat proliferation to a position having fat depression after the radio-frequency irradiation module 130 irradiates radio-frequency energy to at least one of the buttock S1, the breast S2, and the retaining ligament.

Since volumetric heating is possible according to an operation mechanism of the present disclosure, fat proliferation at a position having fat depression can be enhanced by irradiating ultrasound energy corresponding to fat proliferation to a position having fat depression with respect to fat reduction occurring at the position having fat depression when radio-frequency is irradiated through the radio-frequency irradiation module 130.

As another example, the ultrasound irradiation module 110 may further irradiate ultrasound energy corresponding to fat proliferation at a perpendicular or oblique angle with respect to a surface of the buttock S1 or the breast S2.

Accordingly, the present disclosure can efficiently irradiate ultrasound energy corresponding to fat proliferation to the buttock S1 or the breast S2 for each patient. That is, the ultrasound irradiation module 110 can increase irradiation efficiency by irradiating ultrasound energy in an operation similar to pushing the buttock S1 or the breast S2. Accordingly, the ultrasound irradiation module 110 is preferably designed in a structure capable of irradiating ultrasound energy in an operation similar to pushing the buttock S1 or the breast S2.

In embodiments, the ultrasound irradiation module 110 may further irradiate ultrasound energy corresponding to fat proliferation at a perpendicular or oblique angle with respect to a surface of a zygomatic arch.

The present disclosure can efficiently irradiate ultrasound energy corresponding to fat proliferation without affecting a zygomatic bone. That is, the ultrasound irradiation module 110 may not or not substantially affect the zygomatic bone while irradiating ultrasound energy deeply by irradiating ultrasound energy in an operation similar to pushing a bone. Accordingly, the ultrasound irradiation module 110 is preferably designed in a structure capable of irradiating ultrasound energy in an operation similar to pushing a bone.

As another example, the ultrasound irradiation module 110 may further irradiate ultrasound energy corresponding to fat proliferation to at least one of a lower region PD5 of a buttock, a lower region PD9 of a breast, the deep fat layer S11 of a lower region of an eye, and a lower region of a depressed region. In embodiments, the ultrasound irradiation module 110 may be provided in a pen type to irradiate ultrasound energy corresponding to fat proliferation to at least one of the lower region PD5 of the buttock, the lower region PD9 of the breast, the deep fat layer S11 of the lower region of the eye, and the lower region of the depressed region.

Since there may be positions requiring fat proliferation in at least one of the lower region PD5 of the buttock, the lower region PD9 of the breast, the deep fat layer S11 of the lower region of the eye, and the lower region of the depressed region, ultrasound energy corresponding to fat proliferation can be irradiated, thereby enhancing fat proliferation in at least one of the lower region PD5 of the buttock, the lower region PD9 of the breast, and the lower region of the depressed region.

As another example, the processor 122 may be connected to an ultrasound imaging device 20 through a wired connection or a wireless connection. In embodiments, the ultrasound irradiation module 110 may further irradiate ultrasound energy corresponding to fat proliferation to at least one of the buttock S1, the breast S2, and the deep fat layer S11 based on an image of a polymer particle injected into at least one of the buttock S1, the breast S2, and the deep fat layer S11 acquired through the ultrasound imaging device 20. For example, the polymer particle may be a 30 to 40 nm particle or may be an exosome.

In embodiments, after a polymer particle is injected into at least one of the buttock S1, the breast S2, and the deep fat layer S11, ultrasound energy corresponding to fat proliferation may be irradiated to at least one of the buttock S1, the breast S2, and the deep fat layer S11.

In at least some embodiments, ultrasound energy corresponding to fat proliferation is scattered at a position where a polymer particle is injected, ultrasound energy corresponding to fat proliferation can be irradiated to a deeper position, thereby enhancing fat proliferation.

For example, when ultrasound energy corresponding to fat proliferation is irradiated at a focal depth of 3 mm or 4.5 mm at a position where a polymer particle is injected at a depth of about 3 mm, ultrasound energy corresponding to fat proliferation is scattered, so that ultrasound energy corresponding to fat proliferation can be irradiated up to a target depth position of at least one of the buttock S1, the breast S2, and the depressed region or up to the depth position of about 9 mm where the deep fat layer S11 is located, thereby enhancing fat proliferation.

Accordingly, the skin care device according to the present disclosure can enhance fat proliferation of at least one of the buttock S1, the breast S2, the depressed region, and the deep fat layer S11 for each person by irradiating ultrasound energy corresponding to fat proliferation to at least one of the buttock S1, the breast S2, the depressed region, and the deep fat layer S11.

The processor 122 may further control the ultrasound irradiation module 110 such that the ultrasound irradiation module 110 irradiates ultrasound energy corresponding to fat proliferation to at least one of the buttock S1, the breast S2, the depressed region, and the deep fat layer S11.

In embodiments, the processor 122 may control the ultrasound irradiation module 110 to irradiate ultrasound energy corresponding to fat proliferation to fat pads PD1 to PD9 and BPD provided at an irradiation position of at least one of the buttock S1, the breast S2, the depressed region, and the deep fat layer S11 for activation of fat proliferation. The processor 121 may control the ultrasound irradiation module 110 to irradiate ultrasound energy corresponding to fat proliferation to an individual region directly requiring or desiring or benefiting from fat proliferation among irradiation positions of the buttock S1, the breast S2, the depressed region, and the deep fat layer S11 for each person.

In embodiments, when thickness information for each irradiation position of at least one of the buttock S1, the breast S2, the depressed region, and the deep fat layer S11 acquired through an acquisition device 10 is received, the processor 122 may further control the ultrasound irradiation module 110 such that the ultrasound irradiation module 110 irradiates ultrasound energy corresponding to fat proliferation to a target depth of at least one of the buttock S1, the breast S2, the depressed region, and the deep fat layer S11 associated with the thickness information for each irradiation position.

In embodiments, when external shape information of at least one of the buttock S1, the breast S2, and the depressed region acquired through an acquisition device 10 is received, the processor 122 may further determine a position having fat depression within an individual region based on the external shape information of at least one of the buttock S1, the breast S2, and the depressed region. For example, when skin skeletal information acquired through the acquisition device 10 is received, the processor 121 may further determine a position having fat depression within an individual region based on the skin skeletal information. The processor 122 may further control the ultrasound irradiation module 110 such that the ultrasound irradiation module 110 irradiates ultrasound energy corresponding to fat proliferation to the position having fat depression.

In embodiments, when the radio-frequency irradiation module 130 irradiates radio-frequency energy to at least one of the buttock S1, the breast S2, the depressed region, and a retaining ligament, the processor 122 may further control the ultrasound irradiation module 110 such that the ultrasound irradiation module 110 irradiates ultrasound energy corresponding to fat proliferation to a position having fat depression.

In embodiments, the processor 122 may further control an irradiation angle of the ultrasound irradiation module 110 such that the ultrasound irradiation module 110 irradiates ultrasound energy corresponding to fat proliferation at a perpendicular or oblique angle with respect to a surface of at least one of the buttock S1, the breast S2, and the zygomatic arch.

For example, the processor 122 may further control the ultrasound irradiation module 110 such that the ultrasound irradiation module 110 irradiates ultrasound energy corresponding to fat proliferation to at least one of a lower region PD5 of the buttock, a lower region PD9 of the breast, the deep fat layer S11 of the lower region of an eye, and a lower region of the depressed region.

For example, when an image of a polymer particle injected into at least one of the buttock S1, the breast S2, the depressed region, and the deep fat layer S11 acquired through the ultrasound imaging device 20 is received, the processor 122 may further control the ultrasound irradiation module 110 such that the ultrasound irradiation module 110 irradiates ultrasound energy corresponding to fat proliferation to at least one of the buttock S1, the breast S2, the depressed region, and the deep fat layer S11.

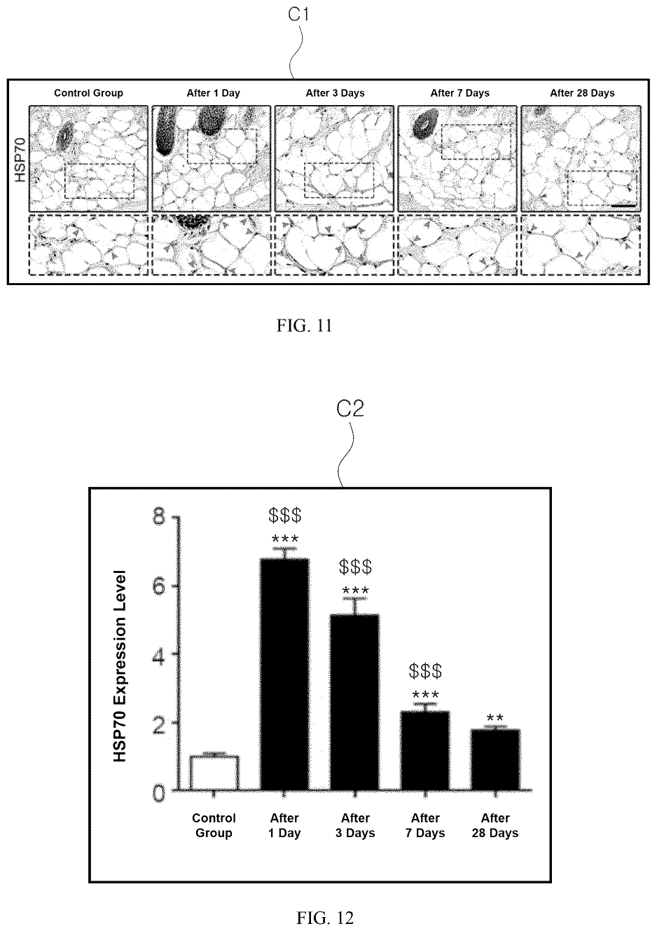

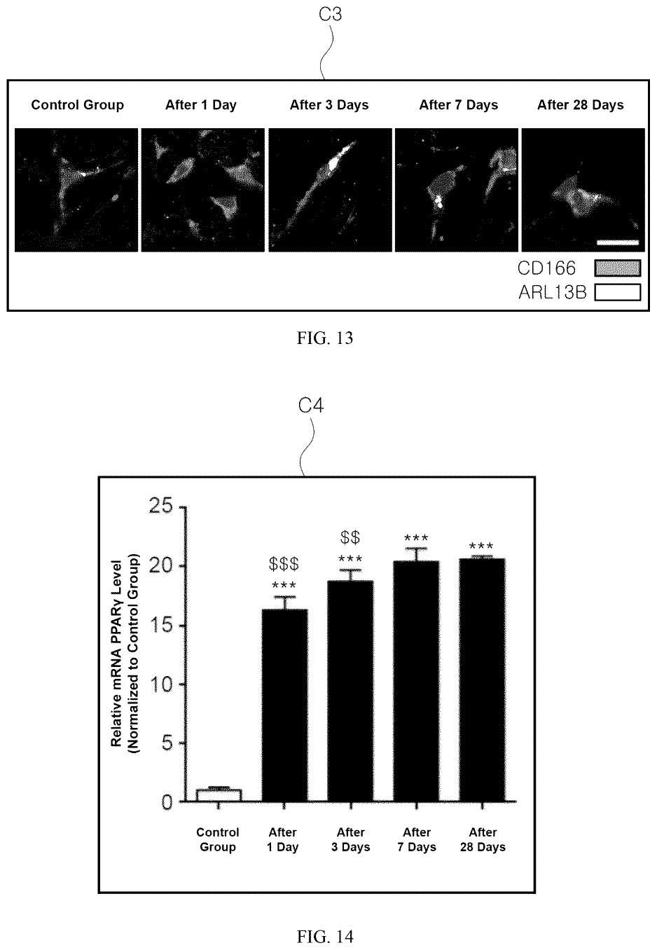

FIGS. 11 and 12 are diagrams illustrating changes in HSP70 expression through cilia regulation of adipose-derived stem cells after ultrasound irradiation of the ultrasound irradiation module of FIG. 2. FIG. 13 is a diagram illustrating a change in cilia length of adipose-derived stem cells after ultrasound irradiation of the ultrasound irradiation module of FIG. 2.

As shown in FIGS. 11 and 12, after ultrasound irradiation to a mini pig, a change in HSP70 expression through cilia regulation of adipose-derived stem cells was analyzed. An HSP70 expression change image C1 and an HSP70 expression change graph C2 were highest after 1 day from ultrasound irradiation compared to a control group in which ultrasound irradiation was not performed, and gradually decreased over time such as after 3 days, after 7 days, and after 28 days.

As shown in FIG. 13, after ultrasound irradiation to a mini pig, a change in cilia length of adipose-derived stem cells was analyzed. A cilia length image C3 was longest after 3 days from ultrasound irradiation in a case of CD166 compared to a control group in which ultrasound irradiation was not performed, and was also longest after 3 days from ultrasound irradiation in a case of ARL13B.

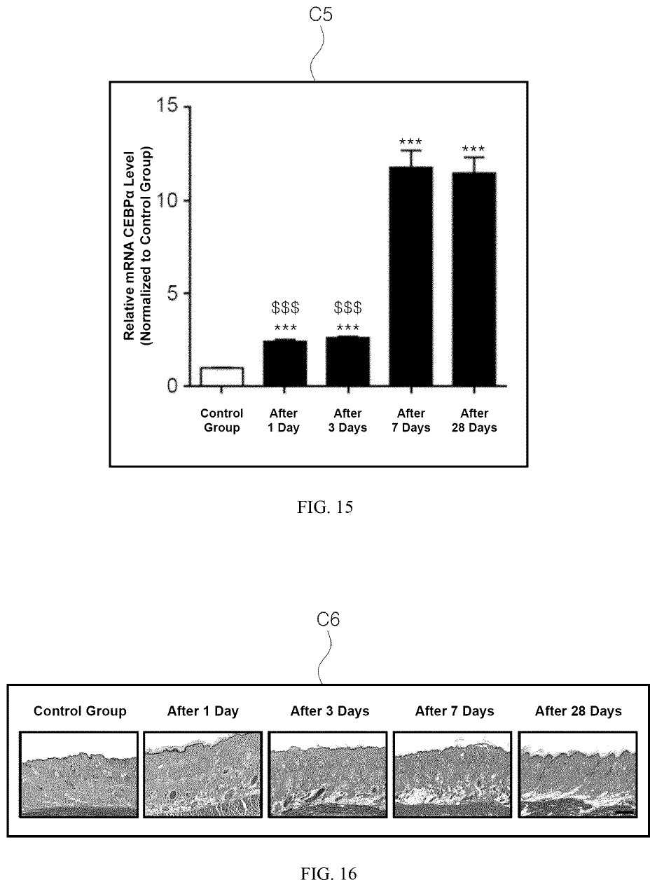

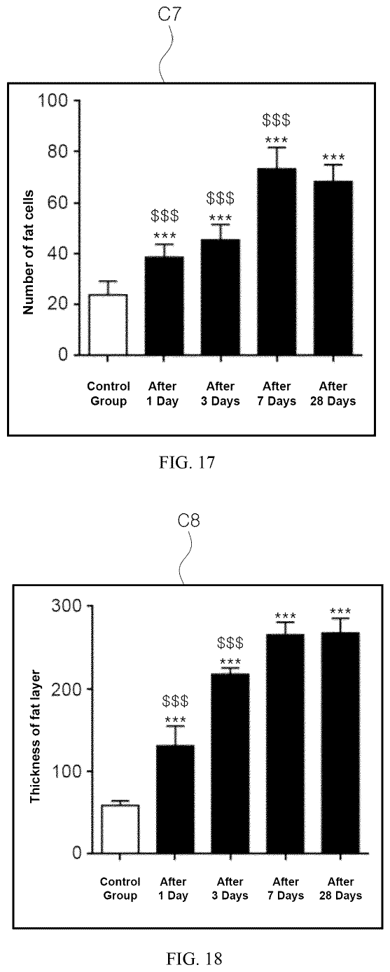

FIGS. 14 and 15 are diagrams illustrating changes in adipogenesis through regulation of cilia length of adipose-derived stem cells after ultrasound irradiation of the ultrasound irradiation module of FIG. 2. FIGS. 16 to 18 are diagrams illustrating changes in the number of adipocytes and thickness of a fat layer through regulation of cilia length of adipose-derived stem cells after ultrasound irradiation of the ultrasound irradiation module of FIG. 2.

As shown in FIGS. 14 and 15, after ultrasound irradiation to a mini pig, a change in adipogenesis through regulation of cilia length of adipose-derived stem cells was analyzed. At this time, an adipogenesis graph C4 was highest after 28 days from ultrasound irradiation in a case of PPARγ compared to a control group in which ultrasound irradiation was not performed. In addition, an adipogenesis graph C5 was highest after 7 days from ultrasound irradiation in a case of CEBPα compared to a control group in which ultrasound irradiation was not performed.

As shown in FIGS. 16 to 18, after ultrasound irradiation to a mini pig, changes in the number of adipocytes and thickness of a fat layer through regulation of cilia length of adipose-derived stem cells were analyzed. At this time, a graph C7 for the number of adipocytes in a fat image C6 was highest after 7 days from ultrasound irradiation compared to a control group in which ultrasound irradiation was not performed. A graph C8 for thickness of a fat layer in the fat image C6 was thickest after 28 days from ultrasound irradiation compared to a control group in which ultrasound irradiation was not performed.

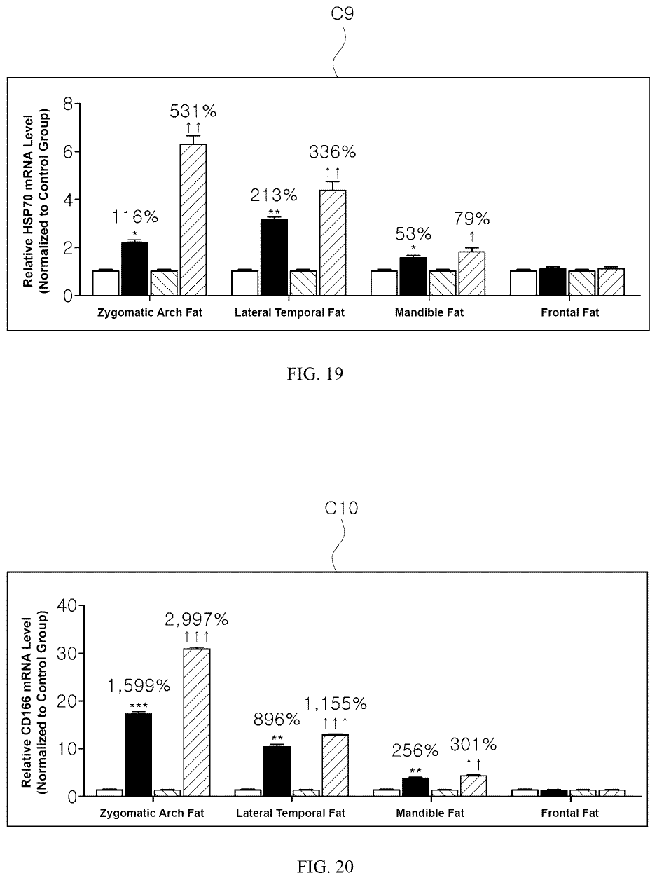

FIG. 19 is a diagram illustrating a change in HSP70 before and after ultrasound irradiation of the ultrasound irradiation module of FIG. 2.

As shown in FIG. 19, an HSP70 change graph C9 showed an improvement effect of 116% when ultrasound irradiation was performed once according to target ultrasound energy on zygomatic arch fat of a mini pig, and showed an improvement effect of 531% when ultrasound irradiation was divided into four times according to target ultrasound energy on the zygomatic arch fat of the mini pig.

The HSP70 change graph C9 showed an improvement effect of 213% when ultrasound irradiation was performed once according to target ultrasound energy on lateral temporal fat of a mini pig, and showed an improvement effect of 336% when ultrasound irradiation was divided into four times according to target ultrasound energy on the lateral temporal fat of the mini pig.

The HSP70 change graph C9 showed an improvement effect of 53% when ultrasound irradiation was performed once according to target ultrasound energy on mandible fat of a mini pig, and showed an improvement effect of 79% when ultrasound irradiation was divided into four times according to target ultrasound energy on the mandible fat of the mini pig.

On the other hand, the HSP70 change graph C9 showed no improvement effect when ultrasound irradiation was performed once according to target ultrasound energy on frontal fat of a mini pig, and also showed no improvement effect when ultrasound irradiation was divided into four times according to target ultrasound energy on the frontal fat of the mini pig.

FIG. 20 is a diagram illustrating a change in CD166 before and after ultrasound irradiation of the ultrasound irradiation module of FIG. 2.

As shown in FIG. 20, a CD166 change graph C10 showed an improvement effect of 1599% when ultrasound irradiation was performed once according to target ultrasound energy on zygomatic arch fat of a mini pig, and showed an improvement effect of 2997% when ultrasound irradiation was divided into four times according to target ultrasound energy on the zygomatic arch fat of the mini pig.

The CD166 change graph C10 showed an improvement effect of 896% when ultrasound irradiation was performed once according to target ultrasound energy on lateral temporal fat of a mini pig, and showed an improvement effect of 1155% when ultrasound irradiation was divided into four times according to target ultrasound energy on the lateral temporal fat of the mini pig.

The CD166 change graph C10 showed an improvement effect of 256% when ultrasound irradiation was performed once according to target ultrasound energy on mandible fat of a mini pig, and showed an improvement effect of 301% when ultrasound irradiation was divided into four times according to target ultrasound energy on the mandible fat of the mini pig.

On the other hand, the CD166 change graph C10 showed no improvement effect when ultrasound irradiation was performed once according to target ultrasound energy on frontal fat of a mini pig, and also showed no improvement effect when ultrasound irradiation was divided into four times according to target ultrasound energy on the frontal fat of the mini pig.

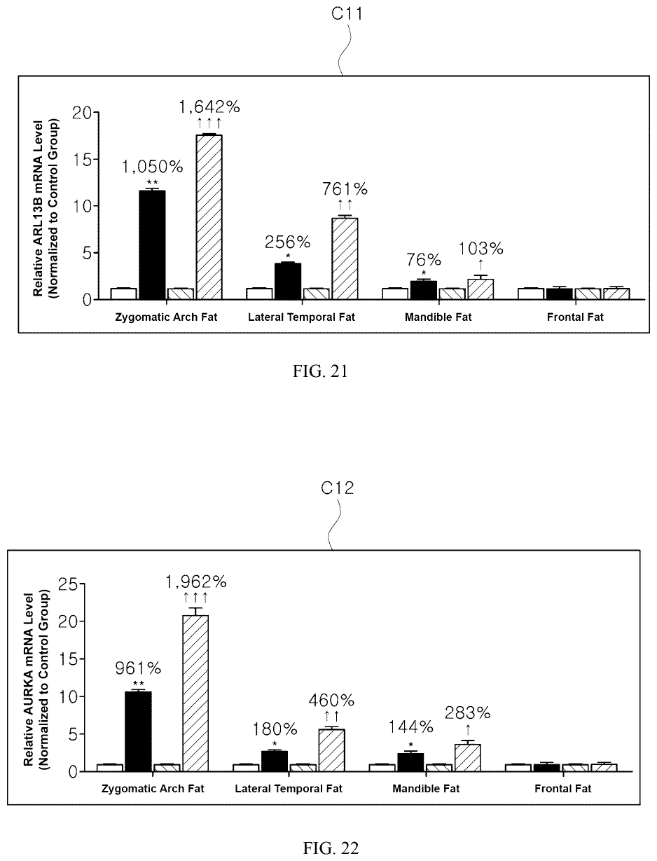

FIG. 21 is a diagram illustrating a change in ARL13B before and after ultrasound irradiation of the ultrasound irradiation module of FIG. 2. FIG. 22 is a diagram illustrating a change in AURKA before and after ultrasound irradiation of the ultrasound irradiation module of FIG. 2.

As shown in FIG. 21, an ARL13B change graph C11 showed an improvement effect of 1050% when ultrasound irradiation was performed once according to target ultrasound energy on zygomatic arch fat of a mini pig, and showed an improvement effect of 1642% when ultrasound irradiation was divided into four times according to target ultrasound energy on the zygomatic arch fat of the mini pig.

The ARL13B change graph C11 showed an improvement effect of 256% when ultrasound irradiation was performed once according to target ultrasound energy on lateral temporal fat of a mini pig, and showed an improvement effect of 761% when ultrasound irradiation was divided into four times according to target ultrasound energy on the lateral temporal fat of the mini pig.

The ARL13B change graph C11 showed an improvement effect of 76% when ultrasound irradiation was performed once according to target ultrasound energy on mandible fat of a mini pig, and showed an improvement effect of 103% when ultrasound irradiation was divided into four times according to target ultrasound energy on the mandible fat of the mini pig.

On the other hand, the ARL13B change graph C11 showed no improvement effect when ultrasound irradiation was performed once according to target ultrasound energy on frontal fat of a mini pig, and also showed no improvement effect when ultrasound irradiation was divided into four times according to target ultrasound energy on the frontal fat of the mini pig.

As shown in FIG. 22, an AURKA change graph C12 showed an improvement effect of 961% when ultrasound irradiation was performed once according to target ultrasound energy on zygomatic arch fat of a mini pig, and showed an improvement effect of 1962% when ultrasound irradiation was divided into four times according to target ultrasound energy on the zygomatic arch fat of the mini pig.

The AURKA change graph C12 showed an improvement effect of 180% when ultrasound irradiation was performed once according to target ultrasound energy on lateral temporal fat of a mini pig, and showed an improvement effect of 460% when ultrasound irradiation was divided into four times according to target ultrasound energy on the lateral temporal fat of the mini pig.

The AURKA change graph C12 showed an improvement effect of 144% when ultrasound irradiation was performed once according to target ultrasound energy on mandible fat of a mini pig, and showed an improvement effect of 283% when ultrasound irradiation was divided into four times according to target ultrasound energy on the mandible fat of the mini pig.

On the other hand, the AURKA change graph C12 showed no improvement effect when ultrasound irradiation was performed once according to target ultrasound energy on frontal fat of a mini pig, and also showed no improvement effect when ultrasound irradiation was divided into four times according to target ultrasound energy on the frontal fat of the mini pig.

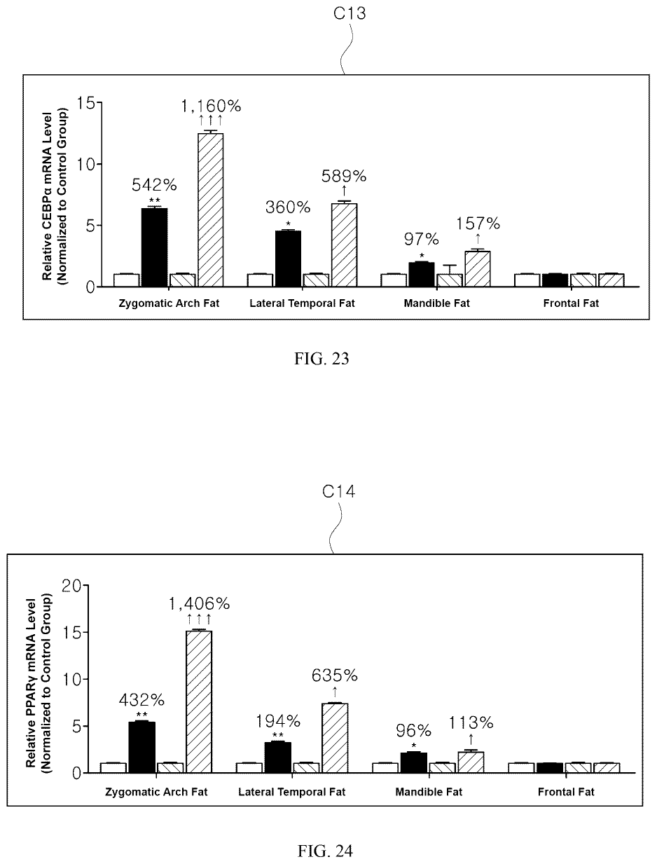

FIG. 23 is a diagram illustrating a change in CEBPα before and after ultrasound irradiation of the ultrasound irradiation module of FIG. 2. FIG. 24 is a diagram illustrating a change in PPARγ before and after ultrasound irradiation of the ultrasound irradiation module of FIG. 2.

As shown in FIG. 23, a CEBPα change graph C13 showed an improvement effect of 542% when ultrasound irradiation was performed once according to target ultrasound energy on zygomatic arch fat of a mini pig, and showed an improvement effect of 1160% when ultrasound irradiation was divided into four times according to target ultrasound energy on the zygomatic arch fat of the mini pig.

The CEBPα change graph C13 showed an improvement effect of 360% when ultrasound irradiation was performed once according to target ultrasound energy on lateral temporal fat of a mini pig, and showed an improvement effect of 589% when ultrasound irradiation was divided into four times according to target ultrasound energy on the lateral temporal fat of the mini pig.

The CEBPα change graph C13 showed an improvement effect of 97% when ultrasound irradiation was performed once according to target ultrasound energy on mandible fat of a mini pig, and showed an improvement effect of 157% when ultrasound irradiation was divided into four times according to target ultrasound energy on the mandible fat of the mini pig.

On the other hand, the CEBPα change graph C13 showed no improvement effect when ultrasound irradiation was performed once according to target ultrasound energy on frontal fat of a mini pig, and also showed no improvement effect when ultrasound irradiation was divided into four times according to target ultrasound energy on the frontal fat of the mini pig.

As shown in FIG. 24, a PPARγ change graph C14 showed an improvement effect of 432% when ultrasound irradiation was performed once according to target ultrasound energy on zygomatic arch fat of a mini pig, and showed an improvement effect of 1406% when ultrasound irradiation was divided into four times according to target ultrasound energy on the zygomatic arch fat of the mini pig.

In addition, the PPARγ change graph C14 showed an improvement effect of 194% when ultrasound irradiation was performed once according to target ultrasound energy on lateral temporal fat of a mini pig, and showed an improvement effect of 635% when ultrasound irradiation was divided into four times according to target ultrasound energy on the lateral temporal fat of the mini pig.

The PPARγ change graph C14 showed an improvement effect of 96% when ultrasound irradiation was performed once according to target ultrasound energy on mandible fat of a mini pig, and showed an improvement effect of 113% when ultrasound irradiation was divided into four times according to target ultrasound energy on the mandible fat of the mini pig.

On the other hand, the PPARγ change graph C14 showed no improvement effect when ultrasound irradiation was performed once according to target ultrasound energy on frontal fat of a mini pig, and also showed no improvement effect when ultrasound irradiation was divided into four times according to target ultrasound energy on the frontal fat of the mini pig.

At least one component may be added or deleted corresponding to performance of the components illustrated in FIGS. 1 to 24. In addition, it will be readily understood by those skilled in the art that mutual positions of the components may be changed corresponding to performance or a structure of the system.

The embodiments disclosed with reference to the accompanying drawings have been described as above. Those skilled in the art to which the present disclosure pertains will understand that the present disclosure may be implemented in forms different from the disclosed embodiments without changing the technical spirit or essential features of the present disclosure. The disclosed embodiments are illustrative and should not be construed as limiting.

According to the above-described means, device and/or methods for solving the problems of the present disclosure, an effect of enhancing fat proliferation of a human body can be provided by irradiating ultrasound energy corresponding to fat proliferation to the human body.

Effects, including effects substantial enough to be clinically significant, are not limited to the above-mentioned effects, and other effects not mentioned above will be clearly understood by those skilled in the art from the description.

1. A skin care device, comprising:
an ultrasound irradiation module to irradiate ultrasound energy to a human body; and
a controller to control operation of the ultrasound irradiation module,
wherein the ultrasound irradiation module is configured to:
irradiate ultrasound energy to the human body to enhance fat proliferation at an irradiation position,
wherein the irradiation position is a position where fat is desired,
pre-irradiate ultrasound energy for activation of the fat proliferation to a fat pad provided at the irradiation position, and
irradiate ultrasound energy to an individual region for the fat proliferation among irradiation positions of the human body.

2. The skin care device of claim 1,
wherein the ultrasound energy for the fat proliferation is 0.01 to 0.2 J.

3. The skin care device of claim 1, wherein:
the controller is further configured to perform wired communication or wireless communication with an acquisition device,
the acquisition device is configured to acquire a preset target depth associated with thickness information for each position of the human body based on preset thickness information for each position of the human body, and
the ultrasound irradiation module is configured to
irradiate ultrasound energy to a target depth associated with the thickness information based on thickness information for a position to which ultrasound is to be irradiated, from the thickness information for each position of the human body acquired from the acquisition device.

4. The skin care device of claim 1, wherein:
the controller is further configured to
perform wired communication or wireless communication with an acquisition device, and
when external shape information of the human body acquired through the acquisition device is received, determine a position having fat depression within an individual region desiring the fat proliferation based on the external shape information of the human body, and
the ultrasound irradiation module is configured to
irradiate ultrasound energy for the fat proliferation to the position having the fat loss.

5. The skin care device of claim 1, wherein:
the device comprises a radio-frequency irradiation module configured to irradiate radio-frequency based on a dielectric electrode to the human body; and
the ultrasound irradiation module is configured to
irradiate ultrasound energy for the fat proliferation to a position having a depression within an individual region desiring the fat proliferation, after the radio-frequency irradiation module irradiates radio-frequency energy to the human body.

6. The skin care device of claim 1,
wherein the ultrasound irradiation module is configured to
irradiate ultrasound energy for the fat proliferation at a perpendicular or inclined angle with respect to a surface of the human body.

7. The skin care device of claim 1, wherein:
the device comprises a processor connected to an ultrasound imaging device through a wired connection or a wireless connection; and
the processor is configured to,
when irradiating the ultrasound energy for the fat proliferation, control the ultrasound imaging device to capture and display at least one of a focal position and a target depth of the human body.

8. The skin care device of claim 1, wherein:
the device comprises a processor connected to an ultrasound imaging device through a wired connection or a wireless connection; and
the ultrasound irradiation module is configured to
irradiate ultrasound energy for the fat proliferation to the human body based on an image of a polymer particle injected into the human body, acquired through the ultrasound imaging device.

9. The skin care device of claim 1, wherein the device is configured for use in connection with at least one of a face, a buttock, a breast, or a depressed region.

10. The skin care device of claim 1,
wherein the ultrasound irradiation module is configured to
irradiate ultrasound energy for the fat proliferation to at least one of a deep fat layer of the face, a lower region of the buttock, a lower region of the breast, or a lower region of the depressed region.

11. The skin care device of claim 1,
wherein the ultrasound irradiation module is configured to
irradiate ultrasound energy for the fat proliferation to a fat pad at an irradiation position of a deep fat layer of a face, and
irradiate ultrasound energy for the fat proliferation to an individual region desiring the fat proliferation among irradiation positions of the deep fat layer.

12. The skin care device of claim 1, wherein:
the device comprises a processor connected to an acquisition device through a wired connection or a wireless connection, and
the processor is configured to,
when skin skeletal information acquired through the acquisition device is received, determine a position having a depression within an individual region requiring the fat proliferation based on the skin skeletal information, and
wherein the ultrasound irradiation module is configured to
irradiate ultrasound energy for the fat proliferation to the position having the depression.

13. The skin care device of claim 1, wherein:
the skin care device comprises a radio-frequency irradiation module configured to irradiate radio-frequency based on a dielectric electrode to a deep fat layer of the face; and
the ultrasound irradiation module is configured to
irradiate ultrasound energy for the fat proliferation to a position having a depression within an individual region desiring the fat proliferation, after the radio-frequency irradiation module irradiates radio-frequency energy to a retaining ligament.

14. The skin care device of claim 1,
wherein the ultrasound irradiation module is configured to
irradiate ultrasound energy for the fat proliferation at a perpendicular or oblique angle with respect to a surface of a zygomatic arch of a face.

15. A skin care device, comprising:
an ultrasound irradiation module to irradiate ultrasound energy to a human body, wherein the ultrasound irradiation module is configured to irradiate ultrasound energy to the human body to enhance fat proliferation at an irradiation position, wherein the irradiation position is a position where fat may provide a benefit, and wherein the irradiation position is one of a deep fat layer of the face, a lower region of the buttock, a lower region of the breast, or a lower region of the depressed region; and
a controller to control operation of the ultrasound irradiation module.

16. The skin care device of claim 15, wherein the ultrasound irradiation module is configured to pre-irradiate ultrasound energy for activation of the fat proliferation to a fat pad provided at the irradiation position.

17. The skin care device of claim 16, wherein the ultrasound irradiation module is configured to irradiate ultrasound energy for the fat proliferation at a perpendicular or inclined angle with respect to a surface of the human body.

18. The skin care device of claim 17, wherein:
the device comprises a processor connected to an ultrasound imaging device through a wired connection or a wireless connection; and
the processor is configured to, when irradiating the ultrasound energy for the fat proliferation. control the ultrasound imaging device to capture and display at least one of a focal position and a target depth of the human body.

19. The skin care device of claim 18, wherein:
the skin care device comprises a radio-frequency irradiation module configured to irradiate radio-frequency based on a dielectric electrode to a deep fat layer of the face; and
the ultrasound irradiation module is configured to irradiate ultrasound energy for the fat proliferation to a position having a depression within an individual region desiring the fat proliferation, after the radio-frequency irradiation module irradiates radio-frequency energy to a retaining ligament.

20. The skin care device of claim 19, wherein the ultrasound irradiation module is configured to irradiate ultrasound energy for the fat proliferation at a perpendicular or oblique angle with respect to a surface of a zygomatic arch of a face.
